(19) United States
(12) Patent Application Publication
(10) Pub. No.: US 2012/0183920 A1
(43) Pub. Date: Jul. 19, 2012

(54) APPARATUS AND METHOD USED IN TEETH WHITENING PROCEDURE

(76) Inventor: Edward Shluper, Ledgewood, NJ (US)

(21) Appl. No.: 13/327,413

(22) Filed: Dec. 15, 2011

Related U.S. Application Data
(60) Provisional application No. 61/426,790, filed on Dec. 23, 2010.

(51) Int. Cl. A61N 5/06; A61C 5/14

(52) U.S. Cl. 433/31; 433/215

(57) ABSTRACT

An apparatus and method for teeth whitening is disclosed that utilized a reflective cheek retractor. The retractor has reflective properties and is flexible enough to be inserted and removed from a patient's mouth. The advantage of the reflective retractor is that the back of the teeth receives better exposure to a light source that activates the teeth whitening substance. A light source or multiple light sources may be disposed on the reflective adaptor to enhance the teeth whitening procedure. The procedure may also be observed using the reflective retractor as an internal mirror for viewing the back of the teeth.

### FIELD OF THE INVENTION

The disclosed embodiments relate generally to an apparatus and method for teeth whitening. More particularly, the disclosed embodiments relate to a reflective oral cheek retractor that provides improved exposure to a light source that activates teeth whitening substances.

### BACKGROUND OF THE INVENTION

In recent times, procedures that whiten teeth have become increasingly popular. There are several methods and tools that are used in teeth whitening techniques. One such technique utilizes a dental tray containing whitening solution or gel. This solution or gel has an active oxidizing ingredient such as hydrogen or carbamide peroxide that provides the whitening effect. The ingredient is applied generally to the patient's teeth (i.e. the top teeth or the bottom teeth) by a dental tray. The tray is kept in place for a certain period of time, and then removed.

Chemical activators in the solution or gel are activated using a lamp to apply light such as ultra-violet or infrared light to accelerate decomposition of the gel or solution and create the whitening effect on the teeth. Generally, the light source is applied to the shape of a full arch over the teeth within the smile zone (L4 to R4). It is preferred that the light source be applied simultaneously to the teeth containing the gel or solution. With current methods, the back of the teeth are difficult to reach with a light source. Placing the light source deep within the mouth gives discomfort to the patient and provides various results.

In addition, if only the front portion of the teeth is exposed to the full whitening procedure, the whitening effect will be less. There remains a need in the field to provide an apparatus and method for whitening both the front and back sides of the teeth for those whitening agent techniques that require exposure to a light source such as ultraviolet or infrared. There also still remains a need in the art for a light source that will activate teeth whitening substances located at the back of a patient's teeth.

### SUMMARY AND OBJECTS OF THE INVENTION

The disclosed apparatus and method provides for a novel way of providing a light source to the back of a patient's teach during a teeth whitening procedure that requires some activation of the teeth whitening substance.

One object of the invention is to provide a reflective apparatus for an external light source to reflect off of and shine light energy upon the back on a patient's teeth to activate a teeth whitening substance. The reflective apparatus may comprise, for example but not limited to, a surface having a coating or film disposed upon an oral cheek retractor apparatus. The retractor may have a portion or portions of the retractor apparatus covered with the reflective material or the entire retractor may be reflective depending on the embodiment.

In another embodiment, the retractor apparatus may comprise arms having reflective panels that reflect a light source energy. The panels may be attachable or integral with the retractor device. Since ease of insertion and removal of the retractor is important to any device placed in a patient's mouth cavity, the apparatus is made of a flexible material that allows insertion and removal without patient discomfort. Examples of materials include but are not limited to plastics, composites, metal alloys, paperboard, and any combination thereof.

Another embodiment includes a light source attached to the retractor. The light source may be an ultraviolet or infrared device. The light source device may be attached, although not so limited, to the mid-section of the retractor between the arm support sections of the retractor. The light source disposed on the retractor may be aimed at the back of the teeth, the reflective portion or portions of the retractor, or both the back of the teeth and the reflective portion or portions of the retractor.

In addition, a plurality of light sources may be used and disposed on the retractor. In one embodiment the light source is located at both the front and back of the retractor. Depending on the embodiment, the light source may be located on the front only, the back only or both the front and back of the retractor to maximize reflective energy to activate the teeth whitening substance on the teeth.

An additional object of the present invention is minimizing the discomfort or trauma associated with using an external light source and/or minimizing the time in the teeth whitening procedure. By utilizing the reflective retractor the back of the teeth not only get better exposure to a light source to activate the teeth whitening substance, an objective already discussed, but also minimize the time needed for the procedure. If a light source is attached to the retractor, the use of an external light source is eliminated as well as the potential discomfort or trauma to the patient.

The foregoing objects are achieved and other features and advantages of the present invention will become more apparent in light of the following detailed description of exemplary embodiments thereof, as illustrated in the accompanying drawings.

### BRIEF DESCRIPTION OF THE DRAWINGS

n the drawings, wherein similar reference characters denote similar elements throughout the several views:

FIG. 1 shows a front view illustration of one embodiment of the apparatus as it is used in a patient's mouth cavity.

FIG. 2 shows a perspective back view without the patient of the apparatus shown in FIG. 1.

FIG. 3 shows a top view of the apparatus shown in FIG. 2.

FIG. 4 shows a side view of the apparatus shown in FIG. 2.

FIG. 5 shows a front view of the apparatus shown in FIG. 2.

FIG. 6 shows a front view of one embodiment of the apparatus shown with a circular mirror reflector.

FIG. 7 shows a front view of one embodiment of the apparatus shown with a polygonal mirror reflector.

FIG. 8 shows a front view of one embodiment of the apparatus shown with a multiple mirror reflectors.

### DETAILED DESCRIPTION

The invention will now be described in detail with reference to the accompanying drawings. The present invention relates to an apparatus and method for improving teeth whitening. The present invention increases exposure of activating light to the teeth whitening substance on the teeth, especially the back of the teeth, and provides a better teeth whitening experience. If the back of the patient's teeth were whiten less due to difficulty in exposing the back of the teeth to a light source, the overall teeth whitening experience would be lessened. The present invention allows a more even distribution of light energy and exposure to activating light energy to the back of a patient's teeth having the whitening substance. By providing better exposure of light energy from inside the back portion of the patient's teeth have a better whitening experience, and therefore the overall front and back of the teeth have a better whitening experience over traditional whitening methods.

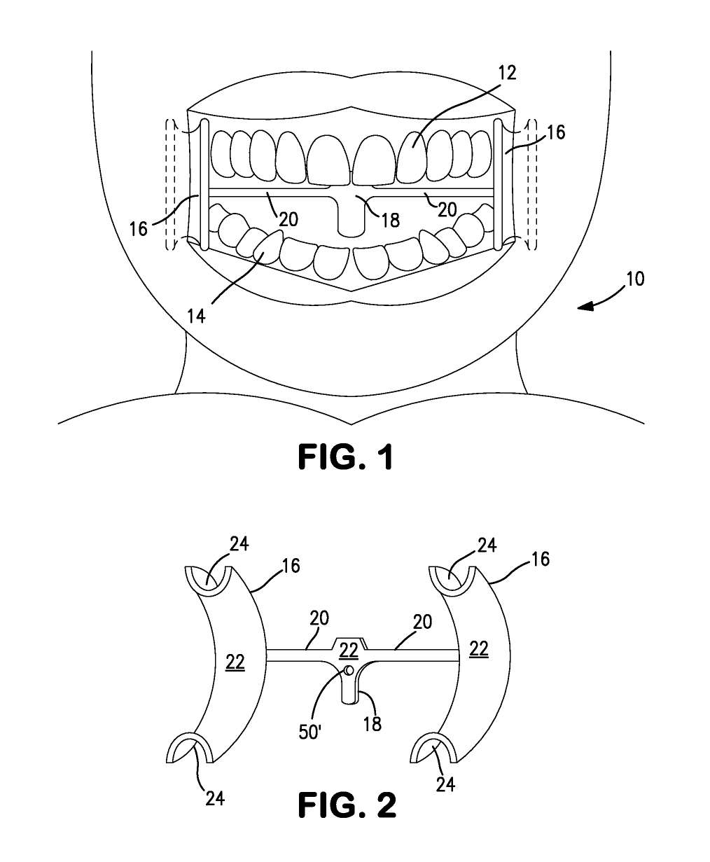

Adverting to the drawings, FIG. 1 illustrates one embodiment of the invention. Shown is a reflective oral cheek retractor 10 inserted in a patient's mouth. The retractor is positioned behind the patient's top teeth 12 and bottom teeth 14. A dental tray carrying the whitening substance is generally applied to both the top teeth and the bottom teeth. Typically the teeth whitening substance required wither ultraviolet (UV) or infrared (IR) light energy to activate the whitening process. The reflective retractor has at least one arm support 20 connected to at least one cheek retractor cup 16. A tab 18 may be used to assist in the insertion and removal of the retractor 10.

The retractor 10 is reflective to allow UV and/or IR light to be reflected off the retractor and onto the teeth, especially the back of the teeth to allow better and more uniform distribution of light energy and a better teeth whitening experience. The retractor may be single use or reusable depending on the implementation. Since the retractor is inserted in the patient's mouth, the retractor is made of a flexible material. Such reflective and flexible materials include, but are not limited to, a metal, a metal alloy, a composite, chrome, tungsten, beryllium, aluminum, plastic, a film, a coating, glass, mirrorbrite, metallic paper or board, mirrored plexiglass, mirrorflex, prismabrite, mylar, and any combinations thereof. Depending on the embodiment, the reflective material may be a film or coating on the retractor and/or an integral part of the retractor. The reflective material may be on the front of the retractor, the back of the retractor, or both the front and back of the retractor depending on the exposure of light desired. In addition, the reflective material may only consist of a portion or portions of the retractor. The reflective material may also be in the form of panels or mirrors (as shown in FIGS. 6-8) disposed on the cheek retractor, such as but not limited to being disposed on the support arms 20 of retractor 10.

FIG. 2 illustrates a back view of reflective retractor 10 and includes a light source 50′. The light source may or may not be included in the reflective retractor depending on the embodiment. As shown in this embodiment the light source may be, but is not limited to, being located on tab 18. The light source may be in the front, back. or both the front and back of the reflective retractor to maximize reflective light to the back of the patient's teeth to activate the whitening substance. There also may be multiple light sources disposed upon the reflective adapter to increase exposure of the light to the teeth whitening substance. For example multiple light sources may be disposed about support arms 20. A number of different lamp designs may be used that are capable of providing the required UV and/or IR light. These lamps generally emit radiation with a wavelength in the range from 340 nm to 600 nm (UV to visible light).

The reflective energy may also reach the front of the patient's teeth to activate the whitening substance as well. Rear surface 22 of the reflective retractor may or may not be reflective depending on the embodiment. Cup surface 24 of cheek cup 16 may also be reflective depending upon the embodiment and desire to reflect the UV or IR light. Again, the UV or IR light may be a source from either an external light source outside the mouth of the patient or an internal light source located inside the mouth of the patient or both. The objective is to provide even distribution of the light source on all surfaces of the teeth to maximize exposure and activation of the teeth whitening substance to maximize the teeth whitening effect. Typically an external light source is used in the teeth whitening procedure. Using an internal light source provides some advantages such as comfort to the patient, and direct exposure of the light energy to the teeth whitening substance. However, the reflective energy from the reflective retractor provides better exposure of the light energy to the back teeth than current traditional methods and will increase the whitening results in the teeth whitening procedure.

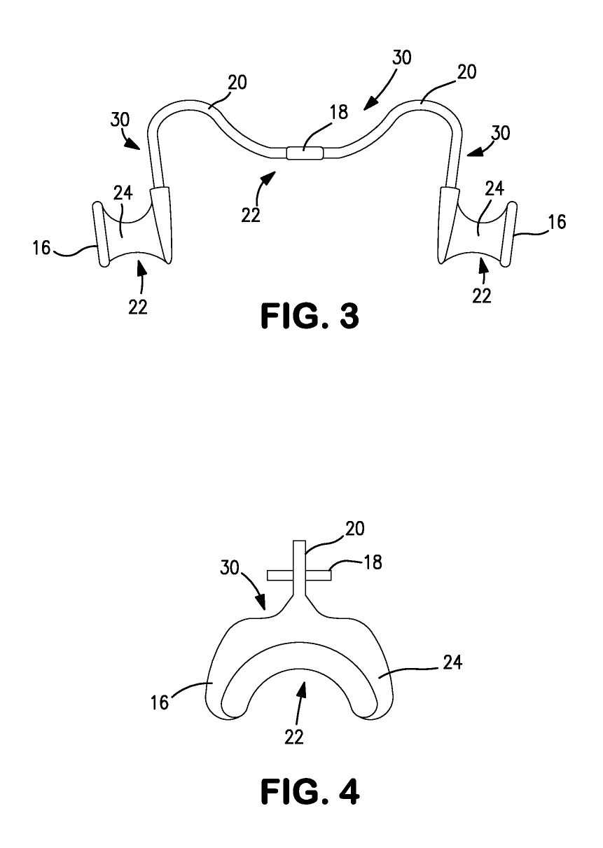

FIG. 3 illustrates a top view of the reflective retractor. Support arms 20 may be designed with geometries to allow flexing so that the retractor may be inserted and removed from a patient's mouth with ease. The bends on support arms 20 such as that shown in FIG. 3 may or may not provide various reflective properties that may or may not be desired depending on the implementation and the end results desired. Front 30 of the reflective retractor that also has a surface may have increased or decreased reflective properties or various refractive indexes to allow a more uniform exposure of the light energy to the teeth. This variation of reflective properties on arms 20 would elevate any variance in reflective powers due to the bending and geometry of the arm sections 20. In the alternative, variation in reflective energy may be desired. For example, the rear portions of the backside of the teeth may get less exposure than the front portions of a patient's teeth and therefore more light energy may be desired in the rear portions.

FIG. 4 illustrates a side view of the reflective retractor. The cheek rest in cheek cup 16 upon surface 24. Surface 24 may or may not have a reflective surface depending on the implementation of the invention. In some cases, it may be desirable to have surface 24 reflective to increase exposure of the light source and activation of the whitening substance.

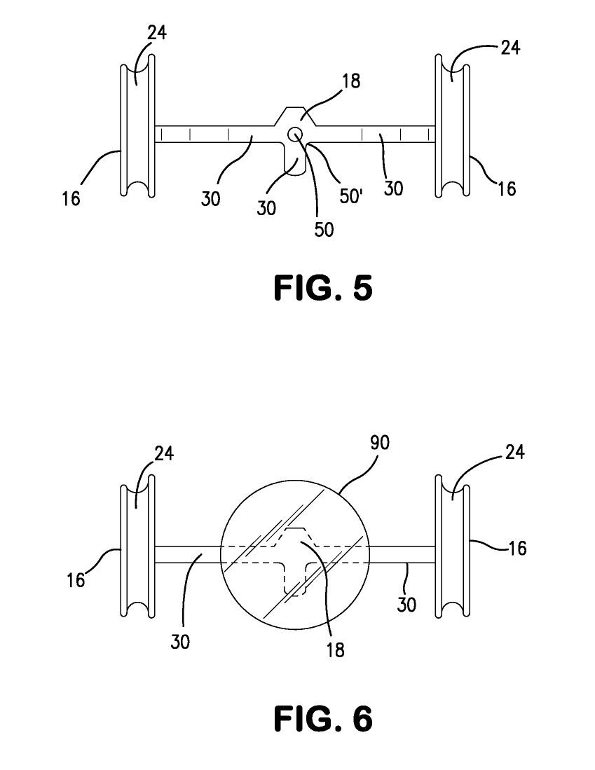

FIG. 5 illustrates the front portion 30 of reflective retractor 10. In this embodiment at least one light source 50 is disposed on tab 18. Again no internal light source is needed for the reflective retractor to reflect energy off an external light source outside the mouth of a patient. On or more light sources may also be disposed about the support arms 20. In addition, it may be desirable to have a light source in the front and back of reflective retractor 10 to allow further exposure of light energy to the back teeth. For example, one light source may have multiple exposure windows 50 and 50′ to allow light from the front and back of the reflective adapter. In addition, 50 and 50′ may be separate light sources depending on the implementation of the invention.

FIGS. 6-8 illustrate the use of a mirror material disposed on the cheek retractor. The use of a reflective material on the retractor in addition to the mirror may or may not be used depending on the embodiment. The use of a reflective material on the support arms for example, in addition to the use of a mirror on the retractor would enhance the whitening procedure by giving a more reflective surface. The mirror may be any shape and size, but preferably sized to fit in a patients mouth.

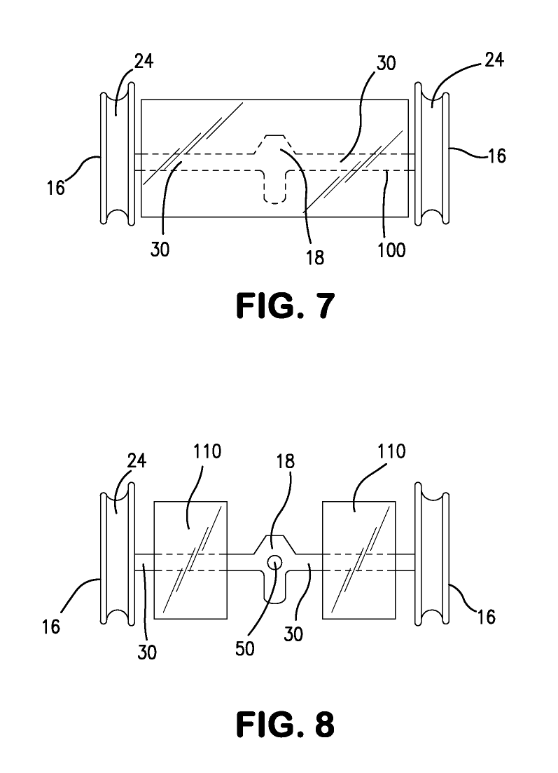

As shown in FIG. 6-7, the mirror may be circular or polygonal such as, but not limited to, a rectangle square and the like. The shape of the mirror may be convex or concave depending on the application needed during the dental procedure. In addition, the use of an interior light source disposed on the cheek retractor may or may not be used. As shown in FIG. 8 multiple mirrors may be incorporated in the cheek retractor. The internal light source, if used on the retractor, preferably does not interfere with any mirrors placed on the cheek retractor. If no mirrors are placed on the cheek retractor, preferably the retractor has a reflective material thereon to reflect a light source to the back of the patients teeth.

In another embodiment, the teeth whitening procedure includes exposure of an external light source that reflects off of reflective retractor 10 and/or mirror disposed on the retractor, and light emanating from the reflective retractor itself. In this embodiment, light is being reflected from the external light source outside the mouth of the patient as well as a light source inside the mouth of the patient.

The advantage of the reflective retractor is that is maximizes exposure of light to the back of the teeth as well as other portions of the teeth to maximize the teeth whitening effect by providing quicker and more uniform activation of the teeth whitening substance applied to the teeth. The improved teeth whitening procedure includes inserting the reflective oral cheek retractor 10 having a reflective material with reflective properties. A light source, whether external, internal, or both in relation to the patient's mouth cavity, is applied and reflected off the oral cheek reflective retractor 10 for light exposure to the teeth and teeth whitening substance.

Applying a teeth whitening substance to the back of teeth is not an issue anymore when utilizing the reflective retractor since light energy will be given to the back of the teeth. Again, depending on the implementation at least one light source may be disposed on the oral cheek reflective retractor to maximize the effect.

Depending on the embodiment, the reflective retractor may have a mirror finish and can be utilized in observing the whitening method by using the reflective oral cheek retractor as a mirror.

Other alternative embodiments or implementations according to the various teachings of the present invention will be understood by those skilled in the art and are achieved without departing from the spirit and scope of the invention. It is therefore intended that the present invention is not limited to the disclosed embodiments described herein but should be defined in accordance with the claims that follow.

I claim:

1. An apparatus used in a teeth whitening procedure, comprising:
an oral cheek retractor having at least one distal arm connected to at least one retracting member for providing access to teeth; and
said oral cheek retractor having a reflective material with reflective properties that allow light from a light source to be reflected to the back of the teeth during a teeth whitening procedure.

2. The apparatus of claim 1 wherein the reflective material is a coating.

3. The apparatus of claim 1, wherein the reflective material is a base material of the oral cheek reflector.

4. The apparatus of claim 1 wherein the reflective material is selected from the group consisting of a metal, a metal alloy, a composite, chrome, tungsten, beryllium, aluminum, plastic, a film, a coating, glass, mirrorbrite, metallic paper or board, mirrored plexiglass, mirrorflex, prismabrite, mylar, and any combinations thereof.

5. The apparatus of claim 1, wherein the light source is disposed on the oral cheek retractor.

6. The apparatus of claim 1, wherein the oral cheek retractor has a front and back and the reflective material is disposed on the front of the retractor.

7. The apparatus of claim 6, wherein the reflective material is disposed on both the front and back of the retractor.

8. The apparatus of claim 1, wherein multiple light sources are disposed on the retractor.

9. The apparatus of claim 5, wherein the light source is either an infrared (IR) or ultraviolet (UV) light source.

10. An apparatus used in a teeth whitening procedure, comprising:
an oral cheek retractor having a distal arm connected to a plurality of retracting members for providing access to teeth; and
a mirror disposed about said arm for allowing ultraviolet or infrared light from a light source to be reflected to the back of the teeth during a teeth whitening procedure.

11. The apparatus of claim 10, further having a light source is disposed on said cheek retractor.

12. The apparatus of claim 10, wherein the oral cheek retractor is a reusable medical device.

13. The apparatus of claim 10, wherein the oral cheek retractor is a disposable medical device.

14. The apparatus of claim 10, wherein the cheek retractor is made of a reflective material.

15. The apparatus of claim 10, wherein the reflective material has flexural properties that allows the oral cheek retractor to be inserted and removed from a mouth.

16. A method for teeth whitening, comprising:
inserting an oral cheek retractor of the type having a reflective material with reflective properties; and
applying a light source to be reflected off the oral cheek retractor for exposure to the teeth.

17. The method of claim 16, further comprising:
applying a teeth whitening substance to the back of teeth.

18. The method of claim 16, further comprising: exposing of the light source to the back of teeth by reflecting light off the oral cheek retractor.

19. The method of claim 16, wherein the light source is on the oral cheek retractor.

20. The method of claim 16, further comprising:
observing the whitening method by using the reflective oral cheek retractor as a mirror.
